SEQUENCE LISTING (4 sequences)

SEQ ID NO: 1 - protein, 4 aa, Homo sapiens
CPSC

SEQ ID NO: 2 - protein, 4 aa, Homo sapiens
CPHC

SEQ ID NO: 3 - protein, 4 aa, Homo sapiens
CPYC

SEQ ID NO: 4 - protein, 4 aa, Homo sapiens
CPFC

(19) United States
(12) Patent Application Publication
(10) Pub. No.: US 2005/0136051 A1
(43) Pub. Date: Jun. 23, 2005

(54) Methods for generating multimeric molecules

(76) Inventor: Bernard Scallon, Wayne, PA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/020,084

(22) Filed: Dec. 22, 2004

Related U.S. Application Data
(60) Provisional application No. 60/531,825, filed on Dec. 22, 2003.

(51) Int. Cl. A61K 39/395; C12P 21/04

(52) U.S. Cl. 424/133.1; 530/387.3; 435/70.21

(57) ABSTRACT

Methods for generating multimeric molecules are disclosed. The methods of the invention are useful for both the in vitro and in vivo formation of multimeric molecules such as bispecific antibodies.

### CROSS-REFERENCE TO RELATED APPLICATIONS

This application claims the benefit of U.S. Provisional Application No. 60/531,825, filed Dec. 22, 2003.

### FIELD OF THE INVENTION

This invention relates to the generation of multimeric molecules such as bispecific antibodies.

### BACKGROUND OF THE INVENTION

The use of monoclonal antibodies (mAbs) as therapeutic reagents has become an effective approach for the treatment of various diseases. In addition, mAbs represent a powerful research tool for gaining a better understanding of the immunopathogenesis of various diseases. IgG isotype mAbs are commonly used as therapeutic reagents and research tools.

Most IgG type antibodies are homodimeric molecules made up of two identical heavy (H) chains and two identical light (L) chains, typically abbreviated H₂L₂. Thus, these molecules are generally bivalent with respect to antigen binding, i.e., both antigen binding (Fab) arms of the IgG molecule have identical binding specificity.

IgG4 isotype heavy chains contain a CPSC (SEQ ID NO: 1) motif in their hinge regions capable of forming either inter- or intra-heavy chain disulfide bonds, i.e., the two Cys residues in the CPSC motif may disulfide bond with the corresponding Cys residues in the other H chain (inter) or the two Cys residues within a given CPSC motif may disulfide bond with each other (intra). It is believed that in vivo isomerase enzymes are capable of converting inter-heavy chain bonds of IgG4 molecules to intra-heavy chain bonds and vice versa (FIG. 1) (Aalberse and Schuurman, Immunology 105, 9-19 (2002)). Accordingly, since the HL pairs in those IgG4 molecules with intra-heavy chain bonds in the hinge region are not covalently associated with each other, they may dissociate into HL monomers that then reassociate with HL monomers derived from other IgG4 molecules forming bispecific, heterodimeric IgG4 molecules (FIG. 2). In a bispecific IgG antibody the two Fabs of the antibody molecule differ in the epitopes that they bind.

Animal studies have demonstrated that administration of bispecific antibody based treatments can destroy tumor cells and improve cancer survival rates. Additionally, bispecific antibodies have been reported to be effective for treatment at lower concentrations than conventional antibodies even when the levels of the target antigen are low. See Kriangkum et al., Biomolecular Engineering 18, 31-40 (2001) and Peipp and Valerius, Biochemical Society Transactions 30, pp. 507-511 (2002).

The potential utility of treating diseases with bispecific antibodies has led to a number of different approaches to produce these molecules. These include use of heterohybridoma cell lines formed by fusing cells of two hybridoma lines that make two distinct Abs, chemical conjugation of two distinct Fab fragments, genetically engineered diabodies, Fos/Jun-mediated dimerization of two Fv domains and other techniques reviewed in Kriangkum et al., supra.

However, many of these approaches to generating bispecific mAbs are labor-intensive and expensive. Also, in many instances bispecific antibodies produced by these methods are inefficiently assembled and the purification of the desired bispecific molecular species from the many undesired molecular species is required. Further, each of the foregoing approaches requires the preparation of bispecific antibodies prior to initiating in vivo studies or undertaking treatments utilizing bispecific antibodies.

Thus, a need exists for methods that can rapidly generate multimeric molecules such as bispecific antibodies both in vitro and in vivo.

### BRIEF DESCRIPTION OF THE DRAWINGS

FIG. 1 is a schematic of the disulfide bonds in IgG1 and IgG4 isotype hinge regions.

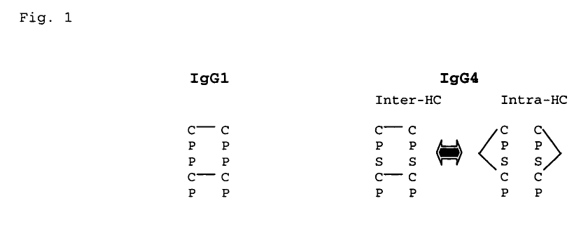

FIG. 2 shows the possible heavy and light chain exchanges between two IgG4 antibodies.

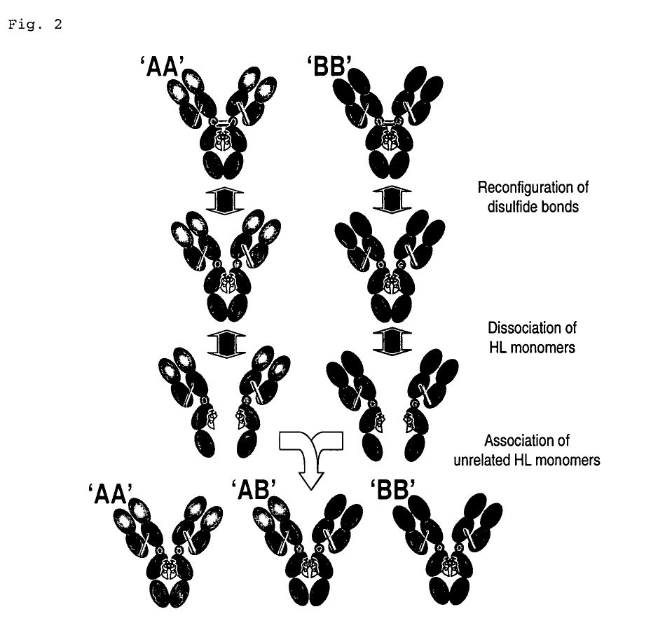

FIG. 3 shows in vitro formation of bispecific antibodies.

FIG. 4 shows inhibition of bispecific antibody formation in vitro.

FIG. 5 shows rapid formation of bispecific antibodies in vitro.

FIG. 6 shows inhibition of bispecific antibody formation in vitro with polyclonal human IgG.

FIG. 7 shows in vivo formation of bispecific antibodies.

FIG. 8 shows a lack of in vivo bispecific antibody formation in mice treated with a single IgG4 antibody.

### SUMMARY OF THE INVENTION

One aspect of the invention is a method for generating a multimeric molecule comprising the steps of providing a first molecule comprising IgG4 antibody heavy chain fragments capable of forming hinge region intra-heavy chain disulfide bonds; providing a second molecule comprising IgG4 antibody heavy chain fragments capable of forming hinge region intra-heavy chain disulfide bonds; mixing the first molecule and second molecule in a solution; and incubating the mixture.

Another aspect of the invention is a method for generating a bispecific antibody comprising the steps of providing a first antibody comprising IgG4 heavy chain fragments capable of forming hinge region intra-heavy chain disulfide bonds; providing a second antibody comprising IgG4 heavy chain fragments capable of forming hinge region intra-heavy chain disulfide bonds; mixing the first antibody and second antibody in a solution; and incubating the mixture.

Another aspect of the invention is a method for generating a multimeric molecule in vivo comprising the steps of providing a first molecule comprising IgG4 heavy chain fragments capable of forming hinge region intra-heavy chain disulfide bonds; providing a second molecule comprising IgG4 heavy chain fragments capable of forming hinge region intra-heavy chain disulfide bonds; administering the first molecule to an animal; and administering the second molecule to the animal.

Another aspect of the invention is a method for generating a bispecific antibody in vivo comprising the steps of providing a first antibody comprising IgG4 heavy chain fragments capable of forming hinge region intra-heavy chain disulfide bonds; providing a second antibody comprising IgG4 heavy chain fragments capable of forming hinge region intra-heavy chain disulfide bonds; administering the first antibody to an animal; and administering the second antibody to the animal.

### DETAILED DESCRIPTION OF THE INVENTION

All publications, including but not limited to patents and patent applications, cited in this specification are herein incorporated by reference as though fully set forth.

The term “antibodies” as used herein is meant in a broad sense and includes immunoglobulin or antibody molecules including polyclonal antibodies, monoclonal antibodies including murine, human, humanized and chimeric monoclonal antibodies and antibody fragments.

Typically, an antibody light chain is linked to an antibody heavy chain by one covalent disulfide bond, while the number of disulfide linkages between the two H chains of an antibody varies between the heavy chains of different immunoglobulin isotypes. Each heavy and light chain also has regularly spaced intra chain disulfide bridges. Each heavy chain has at one end a variable domain (V_{H}) followed by a number of constant domains. Each light chain has a variable domain at one end (V_{L}) and a constant domain at its other end; the constant domain of the light chain is aligned with the first constant domain of the heavy chain and the light chain variable domain is aligned with the variable domain of the heavy chain. Antibody light chains of any vertebrate species can be assigned to one of two clearly distinct types, namely kappa (κ) and lambda (λ), based on the amino acid sequences of their constant domains.

Immunoglobulins can be assigned to five major classes, namely IgA, IgD, IgE, IgG and IgM, depending on the heavy chain constant domain amino acid sequence. IgA and IgG are further sub-classified as the isotypes IgA₁, IgA₂, IgG₁, IgG₂, IgG₃ and IgG₄.

The term “bispecific antibody” as used herein means an antibody that binds two different epitopes.

The term “IgG4 antibody heavy chain fragment” as used herein means a peptide or polypeptide derived from the IgG4 heavy chain such as an entire IgG4 heavy chain or a derivative thereof such as a F(ab′)2 fragment or a modified F(ab′)2-like fragment designed to stabilize the homodimeric F(ab′)2 domain such as can be derived by pepsin or matrix metallproteinase-3 digestion or expressed recombinantly. Further, an IgG4 antibody heavy chain fragment can include an IgG1, IgG2 or IgG3 heavy chain modified to be IgG4-like by having a hinge region sequence motif of CPSC (SEQ ID NO: 1).

The term “mimetibody” as used herein means a protein having the generic formula (I):
(V1(n)-Pep(n)-Flex(n)-V2(n)-pHinge(n)-CH2(n)-CH3(n))(m) (I)
where V1 is at least one portion of an N-terminus of an immunoglobulin variable region, Pep is at least one bioactive peptide that binds to an epitope, Flex is polypeptide that provides structural flexiblity by allowing the mimetibody to have alternative orientations and binding properties, V2 is at least one portion of a C-terminus of an immunoglobulin variable region, pHinge is at least a portion of an immunoglobulin hinge region, CH2 is at least a portion of an immunoglobulin CH2 constant region and CH3 is at least a portion of an immunoglobulin CH3 constant region, where n and m can be an integer between 1 and 10. A mimetibody can mimic properties and functions of different types of immunoglobulin molecules such as IgG1, IgG2, IgG3, IgG4, IgA, IgM, IgD and IgE dependent on the heavy chain constant domain amino acid sequence present in the construct.

The term “monoclonal antibody” (mAb) as used herein means an antibody (or antibody fragment) obtained from a population of substantially homogeneous antibodies. Monoclonal antibodies are highly specific, typically being directed against a single antigenic determinant. The modifier “monoclonal” indicates the substantially homogeneous character of the antibody and does not require production of the antibody by any particular method. For example, murine mAbs can be made by the hybridoma method of Kohler et al., Nature 256: 495 (1975). Chimeric mAbs containing a light chain and heavy chain variable region derived from a donor antibody (typically murine) in association with light and heavy chain constant regions derived from an acceptor antibody (typically another mamammlian species such as human) can be prepared by the method disclosed in U.S. Pat. No. 4,816,567. Humanized mAbs having CDRs derived from a non-human donor immunoglobulin (typically murine) and the remaining immunoglobulin-derived parts of the molecule being derived from one or more human immunoglobulins, optionally having altered framework support residues to preserve binding affinity, can be obtained by the techniques disclosed in Queen et al., Proc. Natl Acad Sci (USA), 86: 10029-10032, (1989) and Hodgson et al., Bio/Technology, 9: 421, (1991).

Fully human mAbs lacking any non-human sequences can be prepared from human immunoglobulin transgenic mice by techniques referenced in, e.g., Lonberg et al., Nature 368: 856-859, (1994); Fishwild et al., Nature Biotechnology 14: 845-851, (1996)′ and Mendez et al., Nature Genetics 15: 146-156, (1997). Human mAbs can also be prepared and optimized from phage display libraries by techniques referenced in, e.g., Knappik et al., J. Mol. Biol. 296: 57-86, (2000) and Krebs et al., J. Immunol. Meth. 254: 67-84, (2001).

The term “multimeric molecules” as used herein and in the claims means molecules that have quaternary structure and are formed by the association of two or more subunits.

The present invention provides methods useful for generating a multimeric molecule or bispecific antibody in vitro or in vivo. In one embodiment of the invention a multimeric molecule is generated in vitro by providing a first molecule comprising an IgG4 antibody heavy chain fragment capable of forming intra-heavy chain hinge region disulfide bonds, providing a second molecule comprising an IgG4 antibody heavy chain fragment capable of forming intra-heavy chain hinge region disulfide bonds, mixing the first and second molecule in a solution, and incubating the mixture.

In another embodiment of the present invention, a bispecific antibody is generated in vitro by providing a first antibody comprising an IgG4 antibody heavy chain fragment capable of forming intra-heavy chain hinge region disulfide bonds, providing a second antibody comprising an IgG4 antibody heavy chain fragment capable of forming intra-heavy chain hinge region disulfide bonds, mixing the first and second molecule in a solution, and incubating the mixture.

In the methods of the invention, the molecules or antibodies may be mixed in a saline solution. In one embodiment, the saline solution may comprise Dulbecco's phosphate buffered saline (D-PBS).

One of ordinary skill in the art can readily determine the amounts of molecules or antibodies to mix in the methods of the invention. For example, such amounts may be those that result in a concentration of each molecule or antibody that is between about 35 μg/ml and about 75 μg/ml.

In the methods of the invention, incubations may be performed across a range of temperatures. Such temperatures will be recognized by those skilled in the art and will include, for example, incubation temperatures at which deleterious physical changes such as denaturation or decomposition do not occur in the mixed molecules or antibodies. In one embodiment, the incubations are performed at room temperature. Typically room temperature is between about 10° C. and about 35° C. An exemplary temperature is about 25° C.

The present invention also provides methods useful for generating a multimeric molecule or bispecific antibody in vivo. In this embodiment of the invention, a multimeric molecule is generated in vivo by providing a first molecule comprising an IgG4 antibody heavy chain fragment capable of forming intra-heavy chain hinge region disulfide bonds, providing a second molecule comprising an IgG4 antibody heavy chain fragment capable of forming intra-heavy chain hinge region disulfide bonds, administering the first molecule to an animal, and administering the second molecule to an animal.

In another embodiment of the invention a bispecific antibody is generated in vivo by providing a first antibody comprising an IgG4 antibody heavy chain fragment capable of forming intra-heavy chain hinge region disulfide bonds, providing a second antibody comprising an IgG4 antibody heavy chain fragment capable of forming intra-heavy chain hinge region disulfide bonds, administering the first molecule to an animal, and administering the second molecule to an animal. The multimeric molecule or bispecific antibody generated in vivo by the method of the invention is useful as a therapeutic agent, diagnostic agent or research reagent.

The bispecific antibody formed in an animal may be purified from the animal's blood and then used for other purposes. An in vivo approach to preparing bispecific antibodies may yield a higher proportion of bispecific antibody than what is likely to be obtained in vitro since disulfide isomerase enzymes in vivo may impart intra-heavy chain disulfide bonds on most all IgG4 molecules such that they all become subject to HL exchange at one time or another. In contrast, only those IgG4 molecules that already contain intra-heavy chain disulfide bonds are likely to participate in HL exchange in vitro.

Alternatively, the yield of bispecific antibodies obtained from in vitro mixing of two IgG4 antibodies may be enhanced by in vitro co-incubation with disulfide isomerase enzymes or some other entity that will convert inter H chain bonds to intra H chain bonds.

The desired enzymatic activity could be obtained from a purified isomerase or from cultured cells expressing an isomerase.

Furthermore, it would be advantageous in some applications to stabilize a desired bispecific antibody molecule so that it is less likely to undergo further HL exchange, particularly in vivo. This could be accomplished by introducing a Cys residue at a strategic site in one IgG4 heavy chain and a Cys residue at a different site in the other IgG4 heavy chain such that a new disulfide bond will be formed between the two heavy chains of the desired IgG4 hybrid but not between the two heavy chains of the original IgG4 antibodies. The method of the invention may also be used for the treatment of animals in need thereof.

In the methods of the invention a molecule or antibody can be administered to an animal. Administration to an animal may be accomplished by injection, ingestion, combinations of administration means or other means readily recognized by those skilled in the art. In one embodiment, molecules or antibodies are administered to an animal that is a mammal. Examples of mammals compatible with the methods of the invention include mice, rats, chimpanzees and humans.

In one embodiment of the invention, at least one molecule or antibody administered to the animal may be administered by injection. Such an injection may occur at different sites or at the same site on an animal. Preferably the injection is made intraperitoneally, but injection or administration may also occur through other routes such as, for example, intramuscularly.

The multimeric molecule or bispecific antibody generated in vitro or in vivo by the methods of the invention is useful as a therapeutic agent, diagnostic agent or research reagent. For example, bispecific antibodies can include a heterodimeric IgG in which one Fab binds to an Fc receptor and the other Fab binds to a tumor-specific antigen. Such bispecific antibodies can specifically bind tumor cells and then bind immune system cells that can kill the tumor cells.

A second example is a heterodimeric IgG in which one Fab binds to T_{reg} cells and the other Fab binds to an antigen associated with inflammation, such as selectin molecules. Such a bispecific antibody could be expected to recruit the inflammation-suppressing T_{reg} cells to sites of inflammation and restore immune homeostasis, a potentially attractive approach for treating autoimmune disorders.

A third example is a heterodimeric IgG in which one Fab binds to a first epitope in a target molecule and the other Fab binds to a second epitope in the same molecule. Bispecific antibodies of this type could prevent conformational changes in proteins such as viral fusion protein or kinases and prevent viral infection or control disease associated kinase signaling.

A fourth example is a heterodimeric IgG in which one Fab binds to a long-lived target such as a red blood cell and the second Fab contains either a particular antigen specificity or an agonist domain. Bispecific antibodies of this type could be used for long-term drug delivery, wherein the second Fab constitutes the drug.

A fifth example is a heterodimeric IgG in which one Fab binds to one diagnostic marker, e.g., on an artificial array of immobilized recombinant antigens, and the other Fab binds to a second diagnostic marker that may be present in a tissue test sample. Such bispecific antibodies of this type could be used to simultaneously test for the presence of two diagnostic markers of interest.

A sixth example is a heterodimeric IgG in which one Fab binds to a specific cell-surface target known to participate in triggering immune responses, e.g., the macrophage mannose receptor, and the other Fab either binds to an antigen to which immune responses are desired or is itself a desired target for an immune response. An anti-idiotype immune response to an antibody may be obtained in this way by preparing an IgG4 version of that antibody and then immunizing animals with a mix of that IgG4 and an IgG4 antibody that binds macrophage mannose receptor. Such a bispecific antibody would be expected to be taken up inside the macrophage, processed and peptide fragments presented to T cells.

Molecules comprising IgG4 antibody heavy chain fragments capable of forming intra heavy chain hinge region disulfide bonds may include, but are not limited to, antibodies, mimetibodies, antibody fragments, small molecule-peptide hybrids or mimetics of these. The heterodimeric products may be derived by mixing any combination of those types of molecules, e.g., one may wish to mix an antibody with a mimetibody to derive a heterdimeric antibody/mimetibody construct.

In the methods of the invention, the IgG4 antibody heavy chain fragments capable of forming intra-heavy chain disulfide bonds include the hinge region sequence motif CPSC (SEQ ID NO: 1). The IgG4 heavy chain fragments can also include variants of an IgG4 antibody heavy chain having at least about 80%, 90% or 95% identity to a known γ4 heavy chain sequence of a given species. Percent identity between two protein sequences can be determined using the BLASTP algorithm (Altschul et al., Nucl. Acids Res. 25, 3389-3402 (1997)) with filtering turned off and all other default settings remaining unchanged. Further, these variants can include hinge region sequence motifs of CPSC (SEQ ID NO: 1), CPHC (SEQ ID NO: 2), CPYC (SEQ ID NO: 3) or CPFC (SEQ ID NO: 4).

The present invention will now be described with reference to the following specific, non-limiting examples.

### EXAMPLE 1

### In Vitro Formation of Bispecific Antibodies

The anti-tumor necrosis factor-α (TNF-α) antibody cA2 G4 and anti-tissue factor (TF) antibody CNTO 859 were used to prepare bispecific anti-TNF-α/anti-TF antibodies. The cA2 G4 antibody is a mouse-human IgG4 chimeric monoclonal antibody against human TNF-α with an intact human IgG4 hinge region. The cA2 G1 antibody is an IgG1 version of cA2 G4 containing a human IgG1 hinge region. The CNTO 859 antibody is a humanized IgG4 monoclonal antibody against human TF with an intact human IgG4 hinge region. The CNTO 859 Fab fragment lacks the IgG4 hinge region and Fc domains.

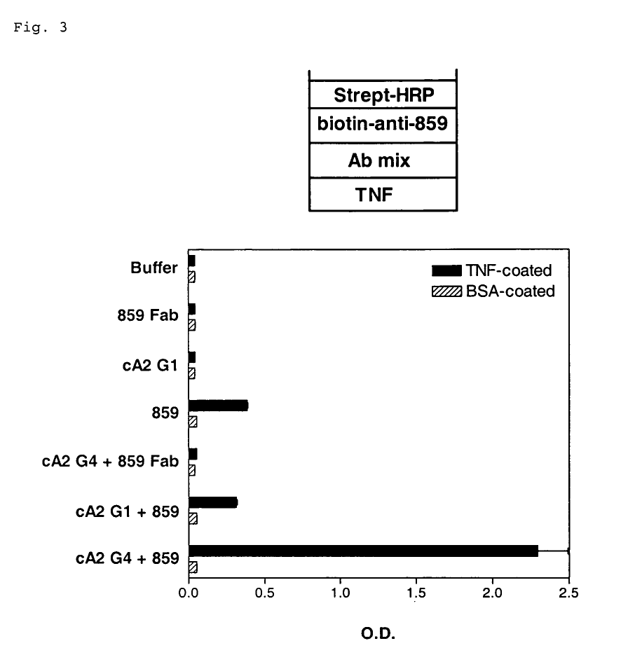

Test samples containing the CNTO 859, cA2 G4, or cA2 G1 antibodies or CNTO 859 Fab as indicated in FIG. 3 were prepared in D-PBS at neutral pH such that the final concentration of each antibody during their coincubation was approximately 71 μg/ml. Test samples were incubated for 1 hr at room temperature.

The test samples were assayed for the formation of bispecific antibodies. Recombinant human TNF-α or bovine serum albumin (BSA) control protein was coated onto 96-well enzyme immunoassay (EIA) plates by placing 50 μl of a 1 μg/ml solution of TNF or BSA in D-PBS in the wells and incubating at room temperature for 1 hr followed by storage at 4° C. Prior to use, plates were washed with a solution of D-PBS containing 1% BSA and 0.05% Tween-20. For assays the antibody test samples were diluted in D-PBS such that the final concentration of each antibody was 4.3 ug/ml. 50 μl of the diluted samples were then added to the TNF-coated plates and the plates were incubated for 1 hr at room temperature.

The plates were again washed with D-PBS containing 1% BSA and 0.05% Tween-20 and then 50 μl of a biotinylated anti-idiotype mAb specific for CNTO 859 (CNTO 4104) was added to the wells to a final concentration of 0.4 μg/ml. Bound CNTO 4104 mAb was detected by adding streptavidin-conjugated horseradish peroxidase (STREPT-HRP) at a concentration of 0.1 μg/ml followed by the chromogenic peroxidase substrate o-phenylenediamine dihydrochloride (OPD). The resulting absorbance of the samples at 490 nm was determined using a Spectramax-340PC plate reader (Molecular Devices Corp., Sunnyvale, Calif.). Each sample was tested in duplicate.

The results in FIG. 3 indicate that the room temperature mixing of the cA2 G4 and CNTO 859 monoclonal antibodies resulted in the in vitro formation of bispecific antibodies able to simultaneously bind to both TNF and the anti-CNTO 859 antibody.

### EXAMPLE 2

### Inhibition of Bispecific Antibody Formation in Vitro

The effect of competitor IgG4 antibodies on the formation of bispecific IgG4 anti-TF/TNF-α antibodies was examined. The α-CD18 IgG4 antibody CNTO 3254 is a mouse-human chimeric monoclonal antibody against human CD18 containing an intact human IgG4 hinge region. The α-CD4 IgG4 antibody CNTO 4132 (or cM-T413) is a mouse-human chimeric monoclonal antibody against human CD4 containing an intact human IgG4 hinge region.

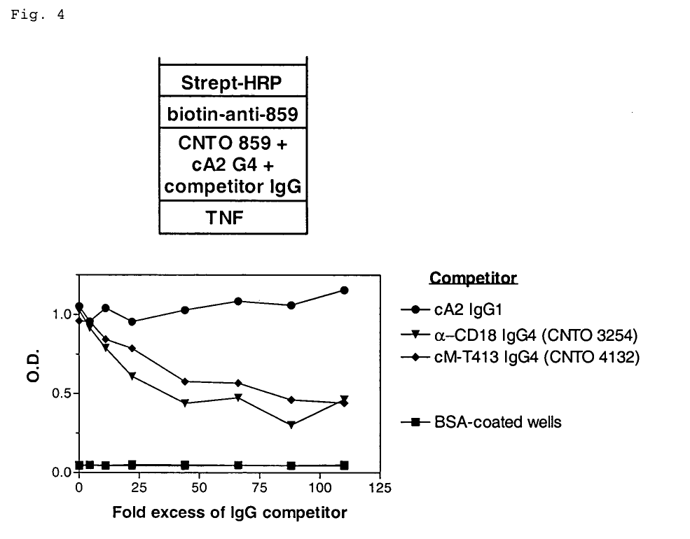

Recombinant human TNF-α or BSA was coated onto 96-well EIA plates by placing 50 μl of a 1 μg/ml solution of TNF-α or BSA in D-PBS in the wells and incubating at room temperature for 1 hr followed by storage at 4° C. The CNTO 859 and cA2 G4 antibodies were then mixed together in D-PBS at neutral pH in the presence of the cA2 IgG1 control, α-CD18 IgG4, and cM-T413 IgG4 competitor antibodies. The final concentration of the CNTO 859 and cA2 G4 antibodies was approximately 41 μg/ml while the competitor antibodies were present in the amounts indicated in FIG. 4. The mixtures of CNTO 859, cA2 G4 and competitor antibodies were then incubated at room temperature for 1 hour.

The mixtures prepared in vitro were then assayed for the formation of bispecific antibodies. Bispecific antibody assays were performed as described in Example 1.

The results in FIG. 4 show that increasing amounts of the α-CD18 IgG4, and cM-T413 IgG4 competitor antibodies inhibited the formation of cA2 G4/CNTO 859 (TNF-α/TF) bispecific antibodies, an indication that those competitor antibodies were also participating in the ongoing HL exchange such that they resulted in less cA2 G4/CNTO 859 hybrids being formed. In contrast, the cA2 G1 antibody, which contains a human IgG1 hinge region, did not cause a decrease in the formation of cA2 G4/859 bispecific antibodies.

### EXAMPLE 3

### Time of Formation of Bispecific Antibodies in Vitro

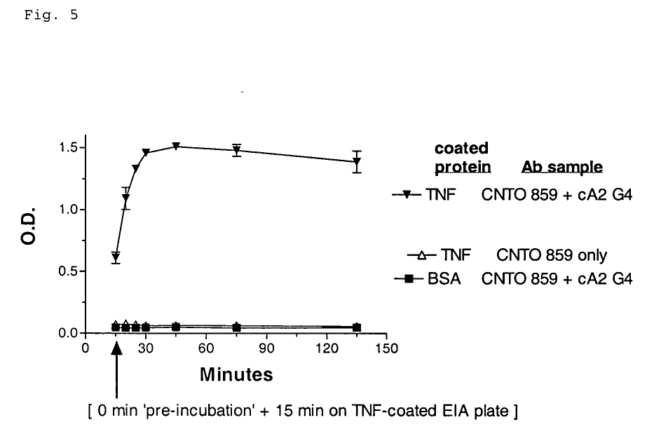

The time course of formation of human TNF-α/TF bispecific antibodies at room temperature was examined. Recombinant human TNF-α or BSA was coated onto 96-well EIA plates by placing 50 μl of a 1 μg/ml solution of TNF or BSA in D-PBS in the wells and incubating at room temperature for 1 hr followed by storage at 4° C. The CNTO 859 and cA2 G4 antibodies were then placed in D-PBS at neutral pH as indicated in FIG. 5. The final concentration of the CNTO 859 and cA2 G4 antibodies was approximately 41 μg/ml. Samples were incubated in vitro at room temperature.

Samples prepared in vitro were assayed for the formation of bispecific antibodies at the time points indicated in FIG. 5. Bispecific antibody assays were performed as described in Example 1.

The results in FIG. 5 show that maximal bispecific antibody formation occurs in vitro approximately 30 minutes after antibody mixing and is detectable as early as 15 minutes after mixing.

### EXAMPLE 4

### Inhibition of Bispecific Antibody Formation in Vitro with Polyclonal Human IgG

Human polyclonal IgG contains different IgG antibody molecules isotypes such as IgG1, IgG2, IgG3, and IgG4 antibody molecules. To follow up on the observations that randomly-selected, irrelevant IgG4 monoclonal antibodies can participate in HL exchange, the more physiologically relevant competitor, total human polyclonal IgG, was tested for its ability to inhibit formation of TNF-α/TF bispecific IgG4 antibodies.

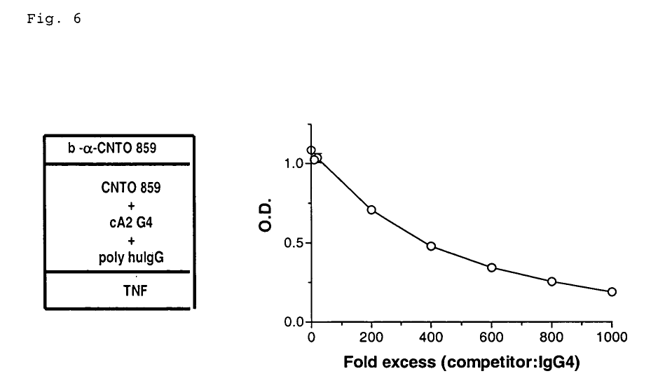

Recombinant human TNF or BSA was coated onto 96-well EIA plates by placing 50 μl of a 1 μg/ml solution of TNF or BSA in D-PBS in the wells and incubating at room temperature for 1 hr followed by storage at 4° C. The CNTO 859 and cA2 G4 antibodies were then mixed together in D-PBS at neutral pH in the presence of human polyclonal IgG competitor antibodies as indicated in FIG. 6. The final concentration of the CNTO 859 and cA2 G4 antibodies was approximately 41 μg/ml while the competitor antibodies were present in the amounts indicated in FIG. 6. Mixtures of the CNTO 859, cA2 G4 and competitor antibodies were then incubated in vitro at room temperature for 1 hour.

The antibody test samples prepared in vitro were then assayed for the formation of bispecific antibodies. Bispecific antibody assays were performed as described in Example 1.

The results in FIG. 6 show that an excess of human polyclonal IgG can reduce the in vitro formation of bispecific antibodies able to simultaneously bind to both TNF and the anti-CNTO 859 antibody and implies that naturally-occurring human IgG4 can also undergo HL exchange reactions.

### EXAMPLE 5

### In Vivo Formation of Bispecific Antibodies

Female CD-1 mice weighing approximately 25 g from (Charles Rivers Laboratories, Raleigh, N.C.) were group housed (6 mice/cage) in plastic filter topped cages and supplied with commercial rodent chow and acidified water ad libitum.

On day 0, mice were given two intraperitoneal (IP) injections as shown in Table 1. The CNTO 859, cA2 G4, and cA2 G1 antibodies were as described in Example 1. Reagents were not mixed prior to injection and were injected separately at two different sites. For each mouse, the two injections were made within a 5 minute period of each other.

TABLE 1
Mouse injection and sampling schedule

| | | Reagent | Reagent | μg | | | Bleed time |
|---|---|---|---|---|---|---|---|
| | | Injection | Injection | reagent | Reagent conc. | Injection | points |
| Group | N | #1 | #2 | per mouse | (mg/ml) | vol (ml) | (hrs) |
| 1 | 2 | PBS | cA2 G4 | 300 | 1.5 | 0.2 + 0.2 | 0.5, 24 |
| 2 | 3 | 859 | PBS | 300 | 1.5 | 0.2 + 0.2 | 0.5, 24 |
| 3 | 3 | 859 | PBS | 300 | 1.5 | 0.2 + 0.2 | 3, 72 |
| 4 | 2 | 859 | cA2 G4 | 300 + 300 | 1.5 + 1.5 | 0.2 + 0.2 | 0.5, 24 |
| 5 | 2 | 859 | cA2 G4 | 300 + 300 | 1.5 + 1.5 | 0.2 + 0.2 | 3, 72 |
| 6 | 3 | 859 | cA2 G1 | 300 + 300 | 1.5 + 1.5 | 0.2 + 0.2 | 0.5, 24 |
| 7 | 3 | 859 | cA2 G1 | 300 + 300 | 1.5 + 1.5 | 0.2 + 0.2 | 3, 72 |
| 8 | 3 | PBS | cA2 G1 | 300 | 1.5 + 1.5 | 0.2 + 0.2 | 0.5, 24 |
| 9 | 3 | PBS | cA2 G1 | 300 | 1.5 + 1.5 | 0.2 + 0.2 | 3, 72 |

Blood samples of approximately 200 μl were collected by retro-orbital bleeds from CO₂ anesthetized mice at 0.5, 3, 24 or 72 hrs after the injections. Each mouse was bled twice. Blood was allowed to sit at room temperature for at least 30 minutes, but not longer than 1 hour. Samples were then centrifuged at 2500 RPM for 20 minutes and the serum was removed. Serum was placed into tubes and stored in the freezer prior to analysis for bispecific antibody formation.

Recombinant human TNF was coated onto 96-well EIA plates by placing 50 μl of a 1 μg/ml solution of TNF or BSA in D-PBS in the wells and incubating at room temperature for 1 hr followed by storage at 4° C. The serum samples were then assayed for the formation of bispecific antibodies. Bispecific antibody assays were performed as described in Example 1 except that undiluted serum samples were assayed.

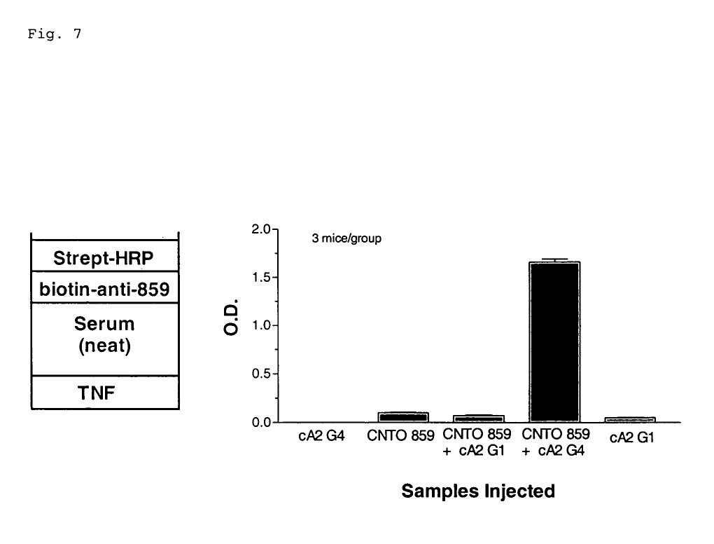

The results indicated that injection of the cA2 G4 and CNTO 859 monoclonal antibodies into mice resulted in the in vivo formation of bispecific antibodies. All blood samples, including those collected just 0.5 hr after IgG4 injection, revealed the presence of the cA2 G4/CNTO 859 bispecific Abs. The results from the 3 hr timepoint are shown in FIG. 7.

### EXAMPLE 6

### Lack of in Vivo Bispecific Antibody Formation in Mice Treated with a Single IgG4 Antibody

Serum samples from mouse treatment groups 1, 2, 3, 4, and 8 of Table 1 in Example 5 were further analyzed to determine if injection of mice with a single IgG4 antibody containing a human IgG4 hinge region would result in bispecific antibody formation (i.e., hybrids of human IgG4 and mouse IgG). Assays for detecting bivalent antibodies capable of binding two molecules of TNF (e.g. cA2 G4 which binds one molecule of TNF on each arm) were performed to make this determination. The assays performed were sensitive to decreases in the number of TNF molecules bound by the antibodies present in a serum sample. Such decreases in TNF binding would occur if HL exchange converted some TNF-specific antibodies capable of binding two TNF molecules into bispecific antibodies capable of specifically binding only one TNF molecule and some second molecule.

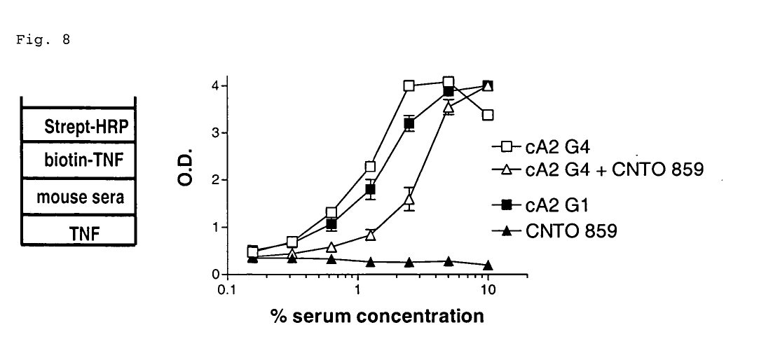

For these assays recombinant TNF was coated onto 96-well EIA plates by placing 50 μl of a 1 μg/ml solution of TNF in D-PBS in the wells and incubating at room temperature for 1 hr followed by storage at 4° C. 50 μl of serum samples corresponding to the 24 hr post injection bleed from mouse treatment groups 1, 2, 3, 4 and 8 were then diluted as indicated in FIG. 8 and added to the TNF-coated plates. Next the plates were incubated for 1 hr at room temperature. Plates were then washed with D-PBS containing 1% BSA and then 50 ul of biotinylated TNF was added to the wells to a final concentration of 0.4 μg/ml. Bound biotinylated TNF was detected by adding STREPT-HRP at a concentration of 0.1 μg/ml followed by the chromogenic peroxidase substrate OPD. The resulting absorbance of the samples at 480 nm was determined using a Spectramax-340PC plate reader (Molecular Devices Corp., Sunnyvale, Calif.).

The results in FIG. 8 indicate that injection of the cA2 G4 antibody alone into mice did not produce a decrease in TNF binding relative to the cA2 G1 control. In contrast injection of both cA2 G4 and the CNTO 859 antibodies resulted in decreased TNF binding apparently as the result of HL exchange. Together the results in FIG. 8 indicate that there is a lack of bispecific antibody formation in mice injected with a single IgG4 antibody containing a human IgG4 hinge region.

The present invention now being fully described, it will be apparent to one of ordinary skill in the art that many changes and modifications can be made thereto without departing from the spirit or scope of the appended claims.

1. A method for generating a multimeric molecule comprising the steps of:
a) providing a first molecule comprising IgG4 antibody heavy chain fragments capable of forming hinge region intra-heavy chain disulfide bonds;
b) providing a second molecule comprising IgG4 antibody heavy chain fragments capable of forming hinge region intra-heavy chain disulfide bonds;
c) mixing the first molecule and second molecule in a solution; and
d) incubating the mixture.

2. A method for generating a bispecific antibody comprising the steps of:
a) providing a first antibody comprising IgG4 heavy chain fragments capable of forming hinge region intra-heavy chain disulfide bonds;
b) providing a second antibody comprising IgG4 heavy chain fragments capable of forming hinge region intra-heavy chain disulfide bonds;
c) mixing the first antibody and second antibody in a solution; and
d) incubating the mixture.

3. The method of claim 1 or 2 wherein the solution comprises a saline solution.

4. The method of claim 1 or 2 wherein the pH of the saline solution is between about 6.0 and about 8.0.

5. The method of claim 1 or 2 wherein the saline solution is D-PBS.

6. The method of claim 1 or 2 wherein the incubation is performed at about room temperature.

7. A method for generating a multimeric molecule in vivo comprising the steps of:
a) providing a first molecule comprising IgG4 heavy chain fragments capable of forming hinge region intra-heavy chain disulfide bonds;
b) providing a second molecule comprising IgG4 heavy chain fragments capable of forming hinge region intra-heavy chain disulfide bonds;
c) administering the first molecule to an animal; and
d) administering the second molecule to the animal.

8. A method for generating a bispecific antibody in vivo comprising the steps of:
a) providing a first antibody comprising IgG4 heavy chain fragments capable of forming hinge region intra-heavy chain disulfide bonds;
b) providing a second antibody comprising IgG4 heavy chain fragments capable of forming hinge region intra-heavy chain disulfide bonds;
c) administering the first antibody to an animal; and
d) administering the second antibody to the animal.

9. The method of claim 7 or 8 wherein the animal is a mammal.

10. The method of claim 7 or 8 wherein the administering of at least one antibody is by injection.

11. The method of claim 1, 2, 7 or 8 wherein the IgG4 derived heavy chain fragments comprise the amino acid sequence CPSC (SEQ ID NO: 1).
